(19) United States
(12) Patent Application Publication
(10) Pub. No.: US 2025/0235644 A1
(43) Pub. Date: Jul. 24, 2025

(54) SYSTEM FOR REMOTE MONITORING OF A NITRIC OXIDE SUPPLY APPARATUS

(71) Applicant: INOSYSTEMS, Antony (FR)

(72) Inventors: Yann BLANDIN, Antony (FR); Stephanie DURAND, Antony (FR); Yasmina SIDIBE, Antony (FR); Anais MCODRUM, Antony (FR); Frederic MARCHAL, Antony (FR)

(21) Appl. No.: 19/032,320

(22) Filed: Jan. 20, 2025

(30) Foreign Application Priority Data
Jan. 19, 2024 (FR) 2400542

(51) Int. Cl. A61M 16/00

(52) U.S. Cl. CPC A61M 16/024; A61M 16/0051; A61M 2202/0007; A61M 2202/0208; A61M 2202/0275; A61M 2205/18; A61M 2205/3553; A61M 2205/502; A61M 2205/52; A61M 2205/583

(57) ABSTRACT

The invention relates to a system (S) for remote monitoring of an NO supply apparatus (1) used to treat patients with an NO-containing gas, comprising means for storing operating data of the apparatus (1) collected during use of the apparatus (1) during successive patient treatments over a given period of time (Dt). The operating data include alarms of different types that are triggered during the period of time in question. A remote server (200) processes the stored operating data and retrieves therefrom information relating to the patient treatments that have been performed and to the operating events that have occurred during said patient treatments. A graphic display (300) displays various items of information relating to the alarms triggered.

### CROSS-REFERENCE TO RELATED APPLICATION

This patent application claims priority to French Application No. 2400542, filed Jan. 19, 2024 and the entire contents of which are incorporated herein by reference.

### TECHNICAL FIELD

The invention relates to a system for remote monitoring of at least one NO supply apparatus, preferably several apparatuses, used for treating patients with an NO-containing gas, in particular an NO/N₂ gas mixture, that is to say an NO-based gas.

### BACKGROUND

Nitric oxide is used for treating persons, i.e. patients, suffering from acute pulmonary arterial hypertension. When inhaled by the patient, NO, called “iNO” or “inhaled NO”, dilates the pulmonary vessels and increases oxygenation by improving gas exchange. These properties are used to treat various medical conditions such as persistent pulmonary hypertension of the newborn (PPHN), acute respiratory distress syndrome (ARDS) observed mainly in adults, or pulmonary hypertension (PH) in heart surgery, observed in adults or children, as described in particular by EP-A-560928, EP-A-1516639 and U.S. Pat. No. 10,201,564.

A gas supply installation for implementation of iNO treatment usually comprises one or more NO/N₂ cylinders containing an NO/N₂ gas mixture of given composition, an NO supply apparatus fed with NO/N₂ mixture, and a medical ventilator supplying a flow of gas containing oxygen (i.e. approximately >20 vol %), such as air, which NO supply apparatus and medical ventilator feed a patient circuit fluidically connected to a respiratory interface (e.g. tracheal intubation tube, mask or the like), which circuit comprises a flow sensor and serves to convey the gas flow. It may comprise other elements, such as a gas humidifier or the like.

The NO supply apparatus and the medical ventilator are fluidically connected to the patient circuit in order to supply it with the NO/N₂ gas mixture and with the flow of oxygen-containing gas, respectively.

The NO/N₂ gas mixture fed to the NO supply apparatus contains a small amount of gaseous NO (e.g. <1000 ppm vol.) diluted in nitrogen (N₂). It is then injected by the NO supply apparatus into the gas flow containing oxygen, typically at least about 20 vol % of oxygen (O₂), such as an N₂/O₂ mixture or air, or even pure oxygen, coming from the medical ventilator, in order to form a final NO/N₂/O₂ gas mixture. The NO/N₂ gas mixture is thus diluted in the oxygen-containing gas flow, and the resulting final gas mixture therefore contains NO, nitrogen and oxygen, or even unavoidable impurities.

The final NO/N₂/O₂ gas mixture is conveyed through the patient circuit and is then administered by inhalation to the patient via the respiratory interface, such as a tracheal intubation tube, breathing mask or the like, supplying the gaseous NO to the airways and/or lungs of the patient to be treated.

The final NO concentration in the final gas mixture (i.e. NO/N₂/O₂) administered to the patient corresponds to a dosage determined by a physician or the like. In general, it is between 1 and 80 ppm by volume (ppmv), typically of the order of 10 to 30 ppmv, depending on the population treated, i.e. neonates, children, adolescents or adults, and on the disease to be treated.

The NO supply apparatus is therefore used to control the amount of NO/N₂ mixture injected into the oxygen-based gas flow coming from the ventilator and to monitor the treatment, in particular the amounts of NO supplied, the NO setpoint values that have been set, the alarm thresholds that have been set (e.g. NO, NO₂, etc.), any alarm triggers or other settings or events that have occurred during the treatment of the patient(s). In other words, the NO supply apparatus makes it possible to ensure supply of NO, and the monitoring of this supply, and to trigger audible and visual alarms in the event of related problems being detected.

When an NO supply apparatus is used in a hospital environment, numerous alarms are triggered that have different levels of criticality, all of them requiring action on the part of the healthcare personnel. Thus, each alarm triggered must be acknowledged by the caregiver, who has to understand the origin of the alarm and, if necessary, implement one or more appropriate corrective measures.

To do this, at present, the healthcare personnel have to record in writing, for example in a register, notebook or similar, the list of all the alarms that have been triggered and also other related information, such as the one or more apparatuses concerned, the one or more patients concerned, the date and time of the triggering, etc., in order to be able to visualize afterwards which alarms have been triggered over the period of time considered, and especially to understand the reasons for these alarm triggers so as to be able to take appropriate corrective measures.

Although the alarms are essential for patient safety, they also create stress for the healthcare personnel and take up time, in particular due to the entries that have to be made in the register.

In addition, these written entries made by the healthcare personnel are sources of error and omission, leading to errors of interpretation, or even to the implementation of inappropriate corrective measures that can impact the effectiveness of future treatments and therefore patient safety.

This is even more true when multiple NO supply apparatuses are used within the same hospital establishment.

Moreover, US2021268221 teaches a device for generating NO from a reactive gas (e.g. N₂ and O₂) by means of electrodes arranged in one or more plasma chambers. A controller serves to regulate the amount of NO produced. A replaceable ventilator cartridge is used to house sensors that monitor flows and may include a memory to store various items of information, such as configuration information, batch or serial numbers, patient or alarm logs, histories, etc. It comprises a monitor for displaying patient curves and alarms that can be remotely connected to a viewing system.

In addition, the user manual for the apparatus for supply of NO by inhalation called INOmax DS_{IR} Plus, 2014 July, allows triggered alarms to be recorded in a memory of the apparatus. The alarm history can be consulted on the apparatus itself.

These apparatuses do not allow remote monitoring of the alarms, especially when several NO apparatuses have to be monitored simultaneously, such as a fleet of apparatuses used within the same hospital establishment.

One problem is therefore to be able to avoid all or some of these problems, in particular those associated with the need to keep a record of the alarms for each NO supply apparatus, in particular in order to improve the effectiveness of iNO treatments and the safety of patients. It will be appreciated that this problem is all the more acute the greater the number of NO supply apparatuses, given that in some hospitals or the like a fleet of several tens of apparatuses may be used to treat patients.

In other words, it is essential to be able to ensure effective monitoring, over time, of one or more NO supply apparatuses used to treat patients in a hospital environment, in particular of a fleet of NO supply apparatuses used within the same hospital establishment, which apparatuses are each likely to trigger alarms during their operation, i.e. when they are being used to treat patients.

### SUMMARY

One solution of the invention relates to a system for remote monitoring of at least one NO supply apparatus used to treat patients with a gas containing NO, in particular an NO/N₂ gas mixture, comprising storage means for storing operating data of the apparatus in question collected during use of the apparatus in successive patient treatments over a given period of time (Dt), each NO supply apparatus being fed with an NO-containing gas, typically an NO/N₂ gas mixture, during said patient treatments, said operating data including different types of alarms triggered during said given period of time (Dt).

Said remote monitoring system comprises:
at least one remote server configured to process the operating data stored by the storage means of the NO supply apparatus and to extract therefrom information relating to the patient treatments that have been performed and to the operational events that have occurred during said patient treatments, and
a graphic display configured to display at least part of said information, the information displayed by the graphic display comprising, for at least one considered time interval (It) of the given period of time (Dt):
   at least some of the most frequently triggered alarms, i.e. the most frequent alarms,
   an occurrence or a number of triggers of at least some of the most frequently triggered alarms,
   a proportion of triggering of at least some of the most frequently triggered alarms,
   an average resolution time of at least some of the most frequently triggered alarms,
   an average number of alarms triggered per treatment, and/or
   an average resolution time of at least some of the most frequently triggered alarms.

In addition, the remote monitoring system further comprises means for selecting the duration of the time interval (It), these means being configured to make it possible for a user to choose a period start date and a period end date, or a period start date and a duration.

Depending on the embodiment considered, the remote monitoring system of the invention can comprise one or more of the following features:
the graphic display is configured to display several different items of information chosen from the abovementioned items of information.
the information displayed by the graphic display includes the 3 to 20 alarms most frequently triggered, i.e. the most frequent alarms, over the time interval (It) in question, preferably the 5 to 15 most frequently triggered alarms, for example the 10 most frequently triggered alarms.
the information displayed by the graphic display comprises what are called critical alarms chosen from among the most frequently triggered alarms, i.e. the most frequent alarms, over the time interval (It) considered, preferably the 3 to 7 critical alarms. The critical alarms correspond to the most frequently triggered alarms having the highest occurrences, i.e. the most numerous alarms, or alarms that have been triggered most often over the time interval (It) considered.
the information displayed by the graphic display comprises the occurrences or numbers of triggers of “critical” alarms.
the information displayed by the graphic display comprises the frequency or proportion (%) of triggers of “critical” alarms.
the graphic display is a colour display.
the graphic display is further configured to display the time interval (It) considered, for example a start date and an end date.
the graphic display is further configured to display, for the time interval (It) considered: a total number of treatments, a number of short treatments, preferably of less than 6 hours, and/or a number of long treatments, preferably of at least 6 hours.
the graphic display is further configured to display, for the time interval (It) considered, 3 to 6 critical alarms corresponding to the 3 to 6 most frequently triggered alarms with the highest occurrences, for example 5 critical alarms.
the graphic display is further configured to display the most frequently triggered alarms and/or critical alarms depending on their occurrence, for example a list starting with the alarm with the highest occurrence and ending with the alarm with the lowest occurrence.
the graphic display is further configured to display the most frequently triggered alarms and/or the critical alarms in one or more display windows displayed on the graphic display.
the means for selecting the duration of the time interval (It) are configured to allow selection of a time interval (It), i.e. a duration, of several weeks, preferably several months, for example from 2 to 12 months, in particular from 3 to 8 months, for example a duration of 1 year.
the NO supply apparatus is fed with gaseous NO coming from one or more NO containers containing said NO-based gas, typically the NO/N₂ gas mixture,
the NO supply apparatus is fed with an NO/N₂ gas mixture coming from one or more NO containers, typically one or more NO cylinders, containing the NO/N₂ gas mixture,
the NO supply apparatus comprises telecommunication means configured to transmit the operating data of the apparatus to said at least one remote server.
the telecommunication means are configured to operate using a mobile network, in particular a network of the 4G, 5G or similar type, or another network.
the telecommunication means comprise transmission means, typically they comprise (at least) a transmitting antenna.
the telecommunication means comprise transmission means configured to transmit data in encrypted and/or analogue form.
said at least one remote server comprises a server of the cloud type, for example the AWS (Amazon Web Service) cloud.
said at least one remote server comprises reception means for receiving the operating data transmitted by the NO supply apparatus.
the types of alarms (i.e. the identified types of alarms) are chosen from:
   an NO concentration “not compliant” alarm, corresponding to an analysed concentration value very different from the set concentration value, for example deviating by at least 30%, preferably at least 40%, more preferably at least 50% from the target dose.
   an NO concentration too low alarm,
   an NO concentration too high alarm,
   an FiO₂ too low alarm,
   an FiO₂ too high alarm,
   a battery fault alarm, i.e. a battery malfunction alarm (e.g. empty, defective, etc.),
   an NO injection line fault alarm,
   a flow measurement line fault alarm, i.e. a malfunction of the flow sensor,
   an alarm indicating a fault in gas flow coming from the ventilator (i.e. air or O₂/N₂ mixture), e.g. no gas flow or, conversely, excessive flow,
   a patient connection fault alarm, for example patient disconnected, or patient connected but without administration of a flow of NO,
   a water trap fault alarm,
   an analysis line fault alarm, i.e. analysis line or sampling line malfunction, and
   a gas source fault alarm (NO, O₂), e.g. non-connection or detection of an empty gas cylinder (i.e. NO cylinder and/or O₂ cylinder), typically an alarm indicating complete absence of a full NO cylinder connected to the NO supply apparatus, i.e. NO cylinder empty alarm.
      preferably, the types of alarms also include:
   an alarm to activate the manual ventilation system (i.e. via a bag valve mask (BVM) connected to the NO supply apparatus),
   an alarm to activate the emergency mode of administration,
   a battery operation alarm, i.e. when the power cord is disconnected or in the event of a failure of the mains supply (i.e. 110V/220V), and/or
   a battery empty or almost empty alarm, for example having a charge of less than 10% of the maximum charge (i.e. fully charged battery).
it further comprises computer processing means configured to receive and process at least some of the processed information supplied by said at least one remote server.
the computer processing means are further configured to control the display of the information displayed by the graphic display.
the computer processing means implement at least one algorithm configured to identify each treatment and to extract therefrom the searched/monitored events, and to process these events in order to determine from them the related alarms.
the graphic display is configured to display at least some of the information relating to each of said NO supply apparatuses.
it is configured to remotely monitor multiple NO supply apparatuses.
it is configured to remotely monitor 2 to 50 NO supply apparatuses used within the same hospital establishment. Indeed, in the largest hospitals, it may be necessary to simultaneously monitor a whole fleet of apparatuses, typically several tens of NO supply apparatuses.
it is configured to remotely monitor at least 5 NO supply apparatuses used within the same hospital, preferably at least 10 apparatuses, more preferably at least 15 to 20 apparatuses.
it comprises selection means configured to allow users to choose or select the apparatus whose information they wish to know, i.e. the alarms, that is to say a given apparatus from a plurality of apparatuses, i.e. a fleet of NO supply apparatuses, used within a hospital.
the selection means comprise one or more keys of a computer keyboard or the like, a touch screen, a mouse and/or similar.
the or each NO supply apparatus is part of a gas administration installation further comprising a medical ventilator for supplying an oxygen-containing gas and a patient circuit fed by the NO supply apparatus with NO-containing gas and by the medical ventilator with oxygen-containing gas.

Depending on the embodiment considered, the or each apparatus or device supplying an NO-containing gas and forming part of the remote monitoring system of the invention can comprise one or more of the following features:
it comprises at least one internal passage, typically a gas circuit comprising several gas passages, for example one or more gas conduits, passages, tubes or the like, for conveying the flow of NO-containing gas, and valve means arranged on said at least one internal passage, for example one or more valves or control valves, in particular one or more solenoid valves or one or more all-or-nothing valves.
it comprises microprocessor-based control means, namely a controller or the like, comprising one or more microprocessors.
the microprocessor-based control means cooperate (at least) with the valve means in order to control the flow of gas in at least part of said internal passage or internal gas circuit.
the control means are configured to control the telecommunication means, in particular to control the transmission of operating data from the apparatus to said at least one remote server.
the microprocessor or microprocessors are arranged on an electronic board.
the storage means comprise a computer storage device, such as a computer memory, for example a flash memory.
the computer memory is arranged on the or an electronic board.
it comprises electrical power supply means, such as an electrical connection to the mains (110/220V).
it comprises a graphical user interface (GUI); preferably, the GUI comprises a display screen and/or selection keys, such as virtual keys displayed on the display screen, i.e. a touch panel screen.

In addition, the or each installation for administering a gas to a patient, i.e. an NO-containing gas, and including the or each apparatus or device for supplying an NO-containing gas comprises:
at least one gas source containing gaseous NO, in particular an NO/N₂ gas mixture, preferably one or more gas containers, such as one or more pressurized gas cylinders,
a gas supply apparatus according to the invention, in particular as described above, fed with NO-containing gas by said at least one gas source, such as the NO/N₂ gas mixture,
a medical ventilator for supplying an oxygen-containing gas, such as air or an O₂/N₂ gas mixture, and
a feed line fed by the gas supply apparatus with NO-containing gas, such as the NO/N₂ gas mixture, and by the medical ventilator with oxygen-containing gas, such as air or the O₂/N₂ mixture.

Depending on the embodiment under consideration, the or each gas administration installation can comprise one or more of the following features:
the (each) medical ventilator, that is to say a ventilation assistance apparatus, is in fluidic communication with the feed line in order to supply said feed line with a respiratory gas containing at least approximately 20 vol % of oxygen, preferably at least approximately 21 vol % of oxygen, in particular air or an N₂/O₂ mixture.
the (each) medical ventilator is a respiratory assistance apparatus supplying the gas at constant pressure or, alternatively, the medical ventilator is an HFO (high-frequency oscillation) ventilator delivering the gas by high-frequency oscillations.
the gas feed line is fed with an NO/N₂ mixture by the (each) gas supply apparatus and with a respiratory gas containing at least approximately 20 vol % of oxygen, preferably at least approximately 21 vol % of oxygen, preferably air or an N₂/O₂ mixture, by the medical ventilator.
the gas feed line is fed with an NO/N₂ mixture by the gas supply apparatus and with a respiratory gas containing oxygen, preferably air or an N₂/O₂ mixture, by the medical ventilator in order to form a final gas mixture for administration to the patient, containing NO, nitrogen and oxygen, or other compounds such as water vapour, and/or impurities such as argon or NO₂ species formed by oxidation of part of the NO.
the final gas mixture to be administered to the patient contains nitrogen, oxygen and NO in a proportion corresponding to a pre-set dose of NO, that is to say a dosage.
the final gas mixture to be administered to the patient contains at least 20 vol % of oxygen, 150 to 1000 ppmv NO and nitrogen, and possibly unavoidable impurities and/or water vapour.
the one or more gas sources contain an NO/N₂ gas mixture containing less than 2000 ppmv of NO, the remainder being nitrogen, preferably less than 1000 ppmv of NO, the remainder being nitrogen.
preferably, the one or more sources of therapeutic gas contain an NO/N₂ mixture containing from 250 to 900 ppmv of NO, the remainder being nitrogen, for example of the order of 800 ppmv of NO, the remainder being nitrogen.
it further comprises a gas humidifier arranged on the gas feed line, preferably downstream of the site where the therapeutic gas supply device is fluidically connected to said gas feed line so as to feed it with therapeutic gas.
it further comprises a line for recovering the gases exhaled by the patient.
the gas feed line and the exhaled gas recovery line are connected at a connecting piece, preferably a Y-piece, and define or form all or part of a patient circuit.
the feed line forms an inspiratory branch of the patient circuit.
the exhaled gas recovery line forms an expiratory branch of the patient circuit.
the NO supply apparatus further comprises a gas analysis line fluidically connected to the gas feed line, i.e. the inspiratory branch.
the gas feed line, i.e. the inspiratory branch, comprises a flow sensor arranged between the ventilator and the site of injection of the NO-containing gas coming from the NO supply apparatus.
the flow sensor is connected to the control means of the NO supply apparatus.
the flow sensor measures the flow of oxygen-containing gas (i.e. air or an N₂/O₂ mixture) delivered by the medical ventilator and circulating in the gas feed line, i.e. the inspiratory branch.
the flow sensor returns gas flow values or signals.
the control means are configured to control the valve means to deliver the gas flow containing NO (e.g. NO/N₂ mixture) at a given rate in order to obtain a dose of NO in the gas feed line corresponding to the desired dose, i.e. the dosage set by the healthcare personnel.
the gas feed line supplies a respiratory interface, for example a breathing mask, a tracheal intubation tube or the like.
the gas feed line, i.e. the inspiratory branch, is fluidically connected to an outlet port of the medical ventilator so as to collect and convey the gas delivered by the medical ventilator.
the exhaled gas recovery line, i.e. the expiratory branch, is fluidically connected to an inlet port of the medical ventilator so as to convey, to the medical ventilator, all or some of the gases exhaled by the patient.
at least one source of therapeutic gas comprises one or more gas containers, particularly one or more pressurized gas cylinders.
the one or more gas containers are equipped with a gas distribution valve with or without an integrated pressure reducer (RDI).
the gas distribution valve is made of copper alloy, such as brass, and/or is equipped with a protective cowl arranged around the gas distribution valve, for example made of polymer material (i.e. plastic), metal or combinations thereof.
the one or more fluid containers are pressurized gas cylinders containing, when full, a gas mixture, in particular NO/N₂, at a pressure of at least 135 to 200 bar abs, or at least 250 to 300 bar abs.
the fluid container has a generally cylindrical shape, in particular ogival.

In general, within the context of the invention:
“ppmv” means parts per million by volume,
“vol %” means percentage by volume.
“NO” denotes nitric oxide.
“NO₂” denotes nitrogen dioxide.
“N₂” denotes nitrogen.
“O₂” designates oxygen.
the terms “concentration”, “dose” and “content” shall be considered as equivalents.
the terms “apparatus” and “device” shall be considered as equivalents.
the terms “supply” and “delivery” shall be considered as equivalents.
the terms “means of/to/for” are considered to be wholly equivalent to and capable of being substituted by the terms “device of/to/for”, for example the term “control means” may be replaced by “control device”, the term “valve means” may be replaced by “valve device”, the “storage means” may be replaced by “storage device”, etc.

### BRIEF DESCRIPTION OF VIEWS OF DRAWINGS

The invention will now be better understood from the following detailed description, which is given by way of non-limiting illustration and with reference to the appended figures, in which:

FIG. 1 schematically illustrates an embodiment of a remote monitoring system according to the invention for monitoring an NO supply apparatus, in particular an NO supply apparatus forming part of a gas administration installation.

FIG. 2 schematically illustrates an embodiment of a gas administration installation comprising an NO supply apparatus that can be remotely monitored by a remote monitoring system according to the invention, such as that of FIG. 1.

FIG. 3 schematically illustrates an example of the displays operated by a remote monitoring system according to the invention, such as that of FIG. 1.

### DETAILED DESCRIPTION

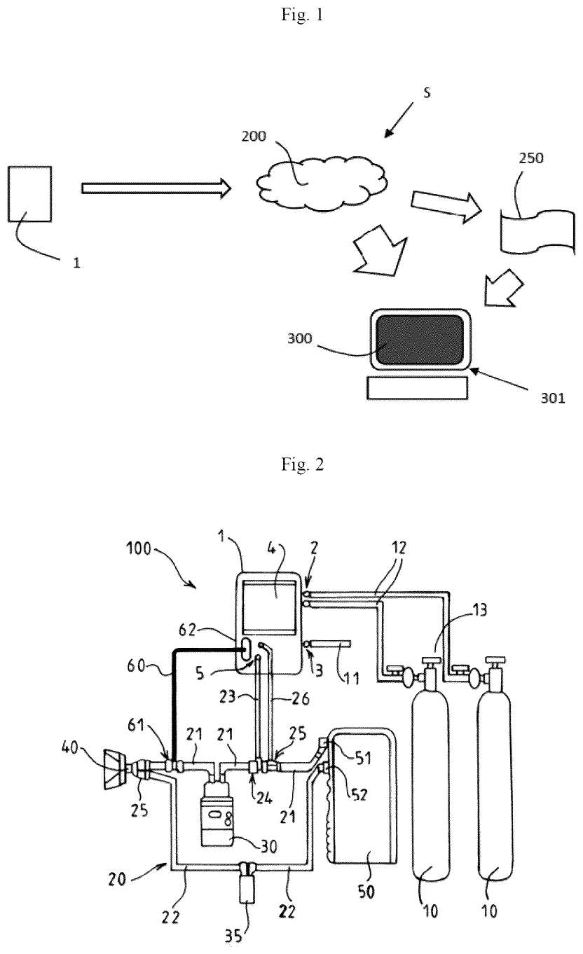

FIG. 2 schematically illustrates an embodiment of a gas administration installation 100 comprising an NO supply apparatus 1, that is to say for supplying a gas mixture based on nitric oxide, which apparatus can be monitored remotely by a remote monitoring system S according to the invention, such as that shown schematically in FIG. 1, as explained below.

More precisely, the installation 100 here comprises two pressurized gas cylinders 10, each containing an NO/N₂ gas mixture, here namely an NO/N₂ gas mixture containing typically between 100 and 1500 ppmv of NO (remainder N₂), for example 450 or 800 ppmv of NO (remainder N₂), or any other suitable concentration, which feed an NO/N₂ mixture to the device or apparatus 1 for supplying, i.e. delivering, NO, making it possible to monitor and control the supply of the NO/N₂ gas mixture.

The gas cylinders 10 are fluidically connected to the NO supply apparatus 1 via gas feed lines 12, such as flexible hoses or conduits or the like, which may be equipped with devices for regulating and/or monitoring the gas pressure, such as gas regulator 13, pressure gauges, etc. The gas feed lines 12 are connected to one or more gas inlets 2 of the NO delivery device 1, which supply an internal gas passage serving to convey the gas within the NO delivery apparatus 1, that is to say in the housing or the outer shell of the apparatus 1.

The NO supply apparatus 1 also comprises an oxygen inlet 3 fluidically connected, via an oxygen feed line 11 such as a flexible hose or the like, to an oxygen source (not shown), for example a pressurized oxygen cylinder or a hospital network, that is to say an oxygen supply line arranged in a hospital building.

The gas administration installation 100 further comprises a medical ventilator 50, that is to say a respiratory assistance apparatus, which supplies an oxygen-based respiratory gas flow, that is to say containing at least about 20 vol % of oxygen, preferably at least 21 vol % of oxygen, such as air or an oxygen/nitrogen (N₂/O₂) mixture.

The medical ventilator 50 and the NO supply apparatus 1 of the installation 100 for administering gas are in fluidic communication with a gas feed line or inspiratory branch 21 of a patient circuit 20. The gas feed line or inspiratory branch 21 serves to convey the gas flow to the patient (not shown), in particular the final gas mixture to be administered to the patient, which flow is formed by mixing the oxygen-based flow (e.g. air or NO/N₂ mixture) from the medical ventilator 50 and the NO-based flow, i.e. the NO/N₂ gas mixture, delivered by the NO supply apparatus 1.

More precisely, the NO supply apparatus 1 delivers or injects the NO/N₂ mixture, for example at 450 or 800 ppmv of NO, into the gas feed line 21 via an injection line or conduit 23 fluidically connecting the internal gas circuit (not visible in FIG. 2) of the NO supply apparatus 1 to the gas feed line 21.

The flow of NO/N₂ supplied by the injection line 23 mixes, at an injection site 24, with the flow of oxygen-based gas (>20% O₂) delivered by the medical ventilator 50 and fed via the upstream portion of the inspiratory branch 21 of the patient circuit 20, so as to obtain the final mixture to be administered to the patient, which essentially contains NO at the desired dosage, nitrogen (N₂) and oxygen (O₂), and possibly inevitable impurities (e.g. argon, CO₂, NO₂, etc,), that is to say a final NO/N₂/O₂ gas mixture.

Preferably, the inspiratory branch 21 further comprises a gas humidifier 30 arranged downstream of the site 24 where NO is injected into the inspiratory branch 21. This makes it possible to humidify the final gas flow before the latter is administered to the patient.

The NO/N₂/O₂ gas mixture is then administered by inhalation, to the patient to be treated, by means of a respiratory interface 40, such as a tracheal intubation tube, a breathing mask or the like, allowing the final gas (i.e. NO/N₂/O₂) to be delivered to the patient's lungs.

A line for recovering the gases exhaled by the patient forms an expiratory branch 22 of the patient circuit 20. It is fluidically connected to the gas feed line or inspiratory branch 21 via a connection piece 25, such as a Y-piece.

The inspiratory branch 21 is fluidically connected upstream to an outlet port 51 of the medical ventilator 50, such as a connector, coupling or the like, so as to recover and convey the oxygen-based gas, typically air or N₂/O₂ mixture, coming from the medical ventilator 50, while the expiratory branch 22 conveying the exhaled gases is fluidically connected to an inlet port 52 of the medical ventilator 50, such as a connector, coupling or the like, so as to return to the medical ventilator 50 all or part of the flow of the gases exhaled by the patient. The expiratory branch 22 can comprise one or more optional components, for example a CO₂ removal device 35, i.e. a CO₂ trap, such as a hot container or the like, used to remove the CO₂ present in the gases exhaled by the patient, or a filter or the like.

A flow rate sensor 25, for example of the hot wire or pressure differential or mass flow type, is arranged on the gas feed line 21, between the ventilator 50 and the humidifier 30, and is connected to the NO delivery apparatus 1 via a flow rate measurement line or lines 26.

The flow sensor 25 serves to measure the flow of gas delivered by the ventilator 50 circulating in the inspiratory branch 21, upstream of the injection site 24 where the injection line 23 is connected and the NO/N₂/O₂ mixture forms. This makes it possible to regulate more efficiently the delivery of the flow of NO (i.e. N₂/O₂) by the NO supply apparatus 1, since the flow rate measurements performed by the flow sensor 25 are returned, via the flow measurement line 26, to control means (not shown) installed in the NO delivery apparatus 1. Typically, the control means comprise an electronic board comprising one or more microprocessors, typically one or more microcontrollers, implementing one or more algorithms.

More specifically, the NO supply apparatus 1 comprises a rigid housing, for example made of polymer, comprising an internal gas circuit (not visible), such as one or more gas conduits or the like, for conveying the NO-based gas flow, i.e. the NO/N₂ mixture, coming from the NO/N₂ mixture cylinders 12, and/or the oxygen flow coming from the oxygen source 11.

The internal gas circuit of the NO supply apparatus 1 fluidically connects the gas inlet (or inlets) 21 of the apparatus 1 to the injection line 23 in order to convey the NO-based gas flow. Valve means (not shown), i.e. one or more valve devices, for example a plurality of solenoid valves arranged in parallel, preferably one or more proportional (solenoid) valves, are arranged on the internal gas circuit of the apparatus 1 in order to control the gas flow which circulates therein in the direction of the injection line 23 and of the injection site 24.

The valve means of the NO supply apparatus 1 are themselves controlled by the control means, i.e. a control device (or devices), also called (micro) controller, arranged in the housing of the NO supply apparatus 1.

The control means make it possible in particular to adjust or control the flow rate of NO-based gas by controlling the valve means, typically by opening or closing said valve or valves, in order to obtain a determined flow rate of NO-based gas, which has been calculated by the control means on the basis of an NO content value set and/or fixed by the user, typically a desired dosage, and as a function of the flow rate of gas, i.e. air or N₂/O₂, delivered by the ventilator 50, which flow rate is measured by the flow sensor 25 arranged on the inspiratory branch 21 and connected to the NO supply apparatus 1, in particular to the control means, by the flow measurement line 26.

The internal gas circuit of the NO supply apparatus 1 can also comprise one or more flow meters arranged upstream and/or downstream of the valve means, in order to determine the flow rate of NO-based gas circulating in the NO supply apparatus 1, in particular to ensure that it complies with the desired flow rate. The flow meter can be of the pressure differential type, the hot-wire type or some other type. It cooperates with the control means in order to provide them with measurements of the flow rate of the NO/N₂ flow. These flow rate measurements are processed by the control means in order to ensure an efficient delivery of NO as a function in particular of the flow rate of O₂ supplied by the medical ventilator 50.

Preferably, the internal gas circuit of the NO supply apparatus 1 can also comprise a pressure regulator, such as a gas regulator or the like, in order to adjust, e.g. reduce, the pressure of the NO-based gas coming from the gas cylinders 10.

Usually, the NO supply apparatus 1 further comprises a graphical user interface (GUI) comprising a graphic display screen 4, preferably a touch screen, i.e. a touch panel, serving to display various information items or data, icons, curves, alerts, etc., and also virtual selection keys and/or panes or windows, in particular for making choices, selections or for entering information, such as desired values (e.g. flow rate, dosage of NO, etc.), or any other information or data useful to the healthcare personnel. The display is preferably in colour, but it can also be in black and white.

The control means of the NO supply apparatus 1 comprise at least one electronic control card and at least one microprocessor-based control unit, typically a microcontroller or the like. The control means also make it possible to carry out calculations and/or to adjust or control all the electromechanical elements of the apparatus 1. More precisely, the control card preferably integrates the control unit and is configured to control and also to analyse and/or process the signals coming from the various components, such as the sensors.

The electrical power for the NO supply apparatus 1, in particular for the components requiring electrical current in order to operate, such as the control means, the graphical display screen 4, etc., is provided conventionally by an electrical current source and/or electrical supply means (not shown), for example a connection to the mains current (110/220V), such as an electrical cord and connection socket, and/or one or more electric, preferably rechargeable, batteries, and/or a current transformer. The electrical power supply to the medical ventilator 50 is ensured in a similar manner, in particular by a connection to the mains current or by an internal battery.

Finally, the installation 100 also comprises a gas sampling line 60 which fluidically connects the inspiratory branch 21 to the NO supply apparatus 1. It is fluidically connected (at 61) to the gas feed line 21, downstream of the injection site 24, namely between the humidifier 30 and the joining piece 25, i.e. the Y-piece, typically in immediate proximity to the joining piece 25, and also to an inlet port 62 of the NO supply device 1, for example a port 62 carried by a connector, coupling or the like, allowing the connection of the gas sampling line 60, such as a flexible hose or the like.

The gas sampling line 60 is used to take gas samples from the inspiratory branch 21 of the patient circuit 20 and to convey them to the NO supply device 1 where they are analysed in an internal gas analyser (not shown), that is to say within a calibration line comprising at least one sensor electrically connected to the control means, in order to verify their conformity. Typically, a calibration line is provided comprising NO₂, NO and O₂ sensors, such as electrochemical cells or the like, which must be calibrated periodically, for example every week.

In particular, it should be verified that the composition of the final gas conforms with that of the desired NO/N₂/O₂ gas mixture to be administered to the patient, in particular in order to ensure that it does not contain excessive amounts of toxic NO₂ species, that its oxygen content is not hypoxic, i.e. <20 vol %), and that its NO content corresponds to the desired dosage, i.e. dose of NO to be administered to the patient that is usually chosen by the healthcare personnel, i.e. physician or the like.

The control means of the apparatus 1 are configured to recover and process, i.e. analyse, the signals coming from the various sensors of the gas analyser and to act in response to these signals, in particular to trigger an alarm in the event of detection of a non-compliant concentration, for example excessive in NO₂, insufficient in O₂ (i.e. hypoxic) or different from the desired NO dosage.

A gas administration installation 100 of this type can be used to administer nitric oxide (iNO), i.e. the final NO/O₂/N₂ mixture obtained, by inhalation to persons, i.e. patients, suffering from acute pulmonary arterial hypertension, in particular to dilate their pulmonary vessels and increase their oxygenation by improving pulmonary gas exchange, in particular to treat persistent pulmonary arterial hypertension of the newborn (PPHN), acute respiratory distress syndrome (ARDS) observed mainly in adults, or pulmonary hypertension (PH) in cardiac surgery, observed in adults or children.

Within a clinical establishment, i.e. hospital or the like, it happens regularly that several NO supply apparatuses 1 are used within a given department and/or in different departments, such as emergency, resuscitation, cardiology departments, etc., that is to say a fleet of NO supply apparatuses 1. It is common to find at least 5 to 10 apparatuses within the same hospital establishment, or even more, for example up to 50 apparatuses in the largest establishments. These apparatuses can be used (at least some of them) simultaneously or in alternation.

However, each of these NO supply apparatuses 1 makes it possible to ensure supply of NO and the monitoring of this supply over time, in particular during successive patient treatments, and to trigger audible and visual alarms if problems are detected during these successive patient treatments.

In order to avoid the healthcare personnel having to record in writing all the alarm triggers and related information for each of the apparatuses, the invention proposes a remote monitoring system S making it possible to monitor one or more NO supply apparatuses 1, in particular an apparatus 1 used in a clinical establishment, such as a hospital or the like, for treating patients with iNO. When they are used, each apparatus is fed with NO as explained above.

Thus, FIG. 1 schematically illustrates an embodiment of such a remote monitoring system according to the invention for monitoring one or more NO supply apparatuses 1, in particular an NO supply apparatus 1 forming part of the gas administration installation 100 of FIG. 2 detailed above.

Generally speaking, in the remote monitoring system S of FIG. 1, each NO supply apparatus 1 is provided with storage means, namely a computer memory or the like, configured to store operating data of each apparatus 1 in question, which data are collected during use or operation thereof, that is to say during the successive patient treatments having taken place during a given period of time Dt, including over long periods of time, for example several weeks or several months, typically over a period of 2 to 18 months, preferably a period of 6 to 12 months.

The recorded operating data comprise in particular the triggering of alarms of different types that have been triggered during the relevant period of time Dt by each apparatus 1, but also all the information related to said triggering, namely the date and time of each triggering of an alarm, the identifier of the apparatus 1 in question, a treatment identification, the hospital site concerned or others.

Moreover, the system S also comprises a remote server (or servers) 200, that is to say a computer server, for example a server of the Cloud type or the like, configured to process the operating data stored by each NO supply apparatus 1 and to extract therefrom the processed information relating to the patient treatments that have been performed and to the operating events that have occurred during said patient treatments, in particular those that have led to the various alarm triggers.

In order to be able to transmit the operating data of each apparatus 1, each NO supply apparatus 1 comprises communication means configured to transmit to the remote computer server 200 which, for its part, comprises receiving means for receiving the operating data transmitted by the NO supply apparatus 1.

More precisely, the system 1 also comprises computer processing means 250, which can be integrated into the server 200 and which are configured to receive and process the information supplied to them by the remote server 200 and then to control the display of the information displayed by a graphic display 300.

The computer processing means 250 comprise one or more (micro) processors implementing one or more algorithms.

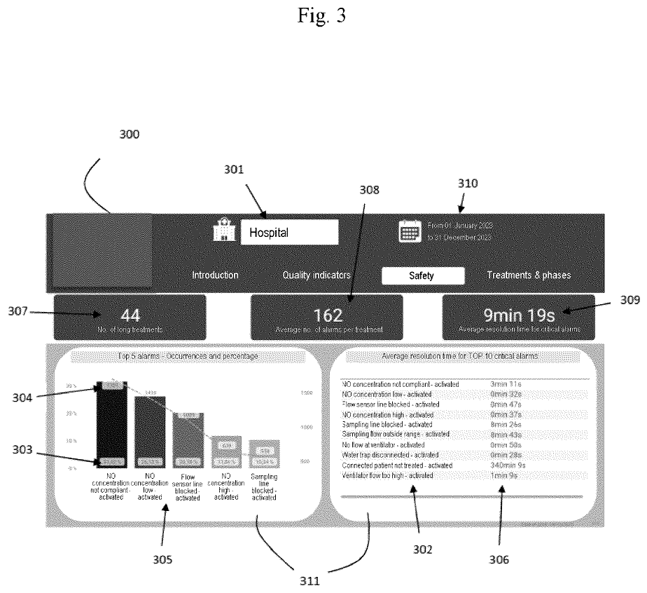

In other words, the remote server 200 cooperates with a graphic display 300, for example that of a computer 301, as illustrated in FIG. 1 and FIG. 3, of a digital tablet or the like, serving to display all or some of the information processed by the remote server 200, in particular by the computer processing means 250, for example the collected and stored information corresponding to a determined time interval (It), for example 1 to 3 months (or another duration) of the given period of time (Dt), for example a period Dt of 12 months.

Preferably, the system S comprises selection means making it possible to select the duration of the time interval (It), for example a duration of several weeks or several months, for example from 2 to 12 months, in particular from 3 to 8 months. This can be done by choosing a period start date and a period end date, or a period start date and a duration, for example 3 months.

In addition, selection means are also provided enabling users to select a particular apparatus whose information they wish to know, i.e. alarms or the like, within the fleet of monitored apparatuses. The selection means comprise, for example, one or more keys or the like.

In particular, according to the invention, the information displayed by the graphic display 300 preferably comprises:
the most frequently triggered alarms 302, that is to say a list of frequent alarms, typically 3 to 15 frequent alarms, for example the 10 most frequently triggered alarms, and preferably the 5 most critical alarms 305 or the most frequently triggered alarms, called critical alarms (or top alarms), among these most frequently triggered alarms 302,
a triggering occurrence 304 (i.e. number) of at least some of the most frequently triggered alarms 302, for example the number of critical alarms 305 triggered,
the proportion (%) 303 of at least some of the most frequently triggered alarms, for example the proportion of the critical alarms 305.
an average resolution time 309 of the most frequently triggered alarms 302, that is to say the time elapsing between an alarm triggering and the acknowledgement of this alarm by the user, for example by pressing an acknowledgement key, by resolving the problem or the like.
an average number of alarms triggered per treatment 308,
an average time (or average duration) of resolution 306 of each of the most frequently triggered alarms 302, and/or
an average time (or average duration) of resolution 309 of the most frequently triggered alarms 302.

Preferably, the graphic display 300 is configured to display several of the items of information in this list, in particular the alarms triggered.

Advantageously, the different types of alarms that can be displayed are for example:
an NO concentration (too) low alarm, e.g. concentration lower than the desired dose,
an NO concentration (too) high alarm, e.g. concentration higher than the desired dose,
an NO concentration “not compliant” alarm, that is to say very far from the desired dose, for example when the concentration deviates by at least 30%, preferably at least 40%, more preferably at least 50% from the expected concentration. This deviation can be prefixed and/or stored.
   an FiO₂ (too) low alarm, e.g. lower than the desired FiO₂,
   an FiO₂ (too) high alarm, e.g. higher than the desired FiO₂,
   a battery malfunction alarm (empty, faulty, etc.),
   an NO injection line fault alarm,
   a flow measurement line fault alarm, i.e. a malfunction of the flow sensor,
   an alarm indicating a fault in the gas flow from the ventilator, e.g. an insufficient or interrupted flow, or too high a flow,
   an alarm indicating patient connected without administration of an NO-based flow,
   an alarm indicating patient not connected, that is to say not receiving NO-based flow,
   an alarm indicating malfunction of water trap, for example disconnected or saturated with moisture,
   an alarm indicating malfunction of analysis line, i.e. the sampling line, and
   an alarm indicating detection of an empty gas cylinder (NO, O₂), i.e. the gas of which has been used up.

Of course, other types of alarms can also be taken into account and displayed on the graphic display 300.

Preferably, the graphic display 300 is also configured to display, for the relevant time interval (It) 310, a total number of treatments, that is to say of patients who have been treated with iNO and/or, among these, the number of short treatments (TC), that is to say treatments having had a duration of less than or equal to a given maximum duration, for example a maximum duration of 6 hours (or another given duration), and/or the number of long treatments (TL) 307, that is to say treatments having had a duration greater than the given maximum duration, for example over 6 hours.

FIG. 3 gives an example of displays performed on the graphic display 300 of a system S according to the invention, such as that of FIG. 1.

It will be seen that, during a given period of time that can be fixed by the user, typically by selecting a period start date and end date 310, here for example between 1 Jan. and 31 Dec. 2023, the following are displayed:
the hospital concerned 301, e.g. the name and address of the hospital,
the 5 critical alarms 302 (called “top 5 alarms”) among the 10 most frequently triggered alarms 305,
the proportion (%) 303 of each of these critical alarms 302.
the number or occurrences 304 of each of these critical alarms 302,
the list of the 10 most frequently triggered alarms and their average resolution time 306,
the number of long treatments 307, for example of at least 6 hours,
the average number of alarms triggered per treatment 308, and
the average duration or average resolution time 309 of the 5 critical alarms 305.

As can be seen here, among the 5 critical alarms 305, the most frequent alarm is the one relating to what is called a “non-compliant” NO concentration, meaning that the measured NO dose is very far from the set dose, that is to say the deviation is at least 50%, for example. This occurs when the healthcare team disconnects the analysis line during treatment; for example, during nebulization/aerosol therapy. Recording this alarm and its recurrence makes it possible to detect this malfunction in the use of the equipment and to correct it by informing the caregiver how to better use the special filters called “for nebulization/aerosol therapy”, whereas the least frequent is the one related to a malfunction of the sampling line (called “blocked”).

In the embodiment shown in FIG. 3, the list of the most frequently triggered alarms 305 and that of the 5 critical alarms 305 are displayed in different display windows 311, namely in this case juxtaposed, preferably large display windows 311 in order to facilitate reading.

In general, the system S of the invention allows remote monitoring of several NO supply apparatuses 1 used within a hospital, and the graphic display 300 makes it possible to display information relating to each of the NO supply apparatuses 1 monitored remotely and used to implement treatment of persons, i.e. patients, with inhaled NO (iNO), especially those suffering from acute pulmonary arterial hypertension, such as persistent pulmonary hypertension of the newborn (PPHN), acute respiratory distress syndrome (ARDS) or pulmonary hypertension (PH) in heart surgery.

What is claimed is:

1. System(S) for remote monitoring of at least one NO supply apparatus (1) used to treat patients with an NO-containing gas, and comprising storage means for storing operating data of the apparatus (1) collected during use of the apparatus (1) during successive patient treatments that have taken place over a given period of time (Dt), the or each NO supply apparatus (1) being fed with NO-containing gas during said patient treatments, said operating data including different types of alarms triggered during said given period of time (Dt),
said system(S) comprising:
at least one remote server (200) configured to process the operating data stored by the storage means of the or each NO supply apparatus (1) and to extract therefrom information relating to the patient treatments that have been performed and to the operating events that have occurred during said patient treatments, and
a graphic display (300) configured to display at least part of said information, the information displayed by the graphic display (300) comprising, for at least one considered time interval (It) of the given period of time (Dt):
at least some of the most frequently triggered alarms (302, 305),
an occurrence or a number of triggers (304) of at least some of the most frequently triggered alarms,
a proportion of triggering (303) of at least some of the most frequently triggered alarms,
an average resolution time (306) of at least some of the most frequently triggered alarms,
an average number (308) of alarms triggered per treatment, and/or
an average resolution time (309) of at least some of the most frequently triggered alarms,
characterized in that it further comprises means for selecting the duration of the time interval (It), these means being configured to make it possible to choose a period start date and a period end date, or a period start date and a duration.

2. System according to claim 1, characterized in that the information displayed by the graphic display (300) comprises the 3 to 15 alarms most frequently triggered over the relevant time interval (It).

3. System according to claim 1, characterized in that the graphic display (300) is further configured to further display:
the relevant time interval (It) (310), and/or
for the relevant time interval (It):
a total number of treatments, a number of short treatments and/or a number of long treatments (307), and/or
critical alarms (305) chosen from the most frequently triggered alarms (302), preferably from 3 to 7 critical alarms (305).

4. System according to claim 1, characterized in that the duration of the time interval (It) is several weeks, preferably several months.

5. System according to claim 1, characterized in that:
the or each NO supply apparatus (1) comprises communication means configured to transmit the operating data of the or each apparatus (1) to said at least one remote server (200), and
said at least one remote server (200) comprises receiving means for receiving the operating data transmitted by the NO supply apparatus (1).

6. System according to claim 1, characterized in that the types of alarms are chosen from:
an NO concentration “not compliant” alarm,
an NO concentration too low alarm,
an NO concentration too high alarm,
an FiO₂ too low alarm,
an FiO₂ too high alarm,
a battery fault alarm,
an NO injection line fault alarm,
a flow measurement line fault alarm,
an alarm indicating a fault in gas flow coming from the ventilator,
a patient connection fault alarm,
a water trap fault alarm,
an analysis line fault alarm, and
a gas (NO, O₂) source fault alarm.

7. System according to claim 1, characterized in that it further comprises computer processing means (250) configured to receive and process at least some of the information provided by said at least one remote server (200), and to control the display of the information displayed by the graphic display (300).

8. System according to claim 7, characterized in that the computer processing means (250) implement at least one algorithm.

9. System according to claim 1, characterized in that it is configured to remotely monitor a plurality of NO supply apparatuses (1).

10. System according to claim 1, characterized in that it is configured to remotely monitor preferably 2 to 50 NO supply apparatuses (1), preferably at least 5 apparatuses.

11. System according to claim 1, characterized in that it comprises selection means configured to allow a user to choose or select a given NO supply apparatus from a plurality of NO supply apparatuses used within the hospital.

12. System according to claim 1, characterized in that the duration of the time interval (It) is 2 to 12 months, in particular 3 to 8 months.

13. System according to claim 1, characterised in that the graphic display (300) is further configured to display the relevant time interval (It), preferably a start date and an end date.

14. System according to claim 3, characterized in that the graphic display (300) is configured to display a frequency or proportion (%) of triggers of critical alarms.

15. System according to claim 1, characterized in that the or each NO supply apparatus (1) is part of a gas administration installation (100) further comprising a medical ventilator (50) for supplying an oxygen-containing gas and a patient circuit (20) fed by the NO supply apparatus (1) with NO-containing gas and by the medical ventilator (50) with oxygen-containing gas.
